(19) United States
(12) Patent Application Publication
(10) Pub. No.: US 2012/0149055 A1
(43) Pub. Date: Jun. 14, 2012

(54) PAPER STRIP FOR DETERMINING MINIMUM INHIBITORY CONCENTRATIONS OF ANTIBIOTICS

(75) Inventor: Silvio Brocco, Roseto Degli Abruzzi (TE) (IT)

(73) Assignee: Liofilchem S.R.L., Roseto Degli Abruzzi (TE) (IT)

(21) Appl. No.: 13/392,308

(22) PCT Filed: Sep. 14, 2010
(86) PCT No.: PCT/EP2010/005644

§ 371 Date: Feb. 24, 2012

(30) Foreign Application Priority Data
Sep. 21, 2009 (IT) AN2009A000064

(51) Int. Cl. C12Q 1/18; C12M 1/34

(52) U.S. Cl. 435/32; 435/287.1

(57) ABSTRACT

The present invention concerns the sector of chemical, clinical, bacteriological and immunological analyses and more specifically it concerns the use of a strip of impregnated paper in order to determine, in the simplest, most economical and most accurate way, the correct Minimum Inhibitory Concentration (M.I.C.) of antibiotic molecules to be administered to patients as claimed in patent No. EP 0 157 071.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention concerns the sector of chemical, clinical, bacteriological and immunological analyses and more specifically it concerns the use of a strip of impregnated paper in order to determine, in the simplest, most economical and most accurate way, the correct Minimum Inhibitory Concentration (M.I.C.) of antibiotic molecules to be administered to patients as claimed in patent No. EP 0 157 071.

2. Description of Related Art Including Information Disclosed Under 37 CFR 1.97 and 37 CFR 1.98

One of the main objectives of medicine is the determination of the correct dosage of antibiotics required both to obtain the greatest effect in order to eradicate the infection and to prevent large doses of antibiotics from having adverse reactions for the patient.

Initially, this problem was resolved in medicine by means of the CLSI (Clinical and Laboratory Standard Institute) standardized microdilution method for dosing the antibiotic with progressively decreasing concentrations. The problem with this method is that it takes a very long time to determine the Minimum Inhibitory Concentration (M.I.C), due to the need to carry out numerous manual laboratory tests in order to identify the correct dose of antibiotic to be administered to the patient.

In order to remedy this problem, a patent exists in prior art, European Patent No. EP 0 157 071, application No. 84850107.8 of 3 Apr. 1984, entitled “Method and device for producing varying concentration patterns of chemically or biologically active substances”, the twenty-year term of which has expired, and which claimed a new method for the determination of the Minimum Inhibitory Concentration (M.I.C.) to be administered to the patient, using a graded carrier with a scale of concentrations of one or more clinically or biologically active substances, so that the substance on this carrier is transferred onto the surface of a culture medium containing bacteria taken from the patient, making it possible to determine the Minimum Inhibitory Concentration (M.I.C).

On expiry of patent EP 0 157 071, the English company OXOID Ltd. launched the M.I.C. Evaluator (M.I.C.E.) on the market. This product is based on the same principle as the aforementioned patent, using a polymer strip graded with a concentration scale of numerous clinically or biologically active substances.

Both in the patent EP 0 157 071 and for the M.I.C Evaluator manufactured by OXOID, the carriers are made of plastic and are difficult to remove from the wrapping which contains them due to the fact that they are very thin, making them more sensitive to the electrostatic attraction forces typical of thin plastic layers and, in order to overcome this problem, the use of specific and expensive equipment is required.

Another problem is that the plastic material of which the carrier is made is impermeable to air. As a result incorrect applications to the microbial culture medium can create air bubbles which may not be visible to the naked eye but which could invalidate the test, or render it inaccurate.

A further problem of the plastic carrier is that as soon as it comes into contact with the agar surface of the microbial culture medium, it immediately starts releasing the antibiotics with which it is impregnated, thereby making its correct repositioning on the microbial culture medium impossible.

Other earlier patents, such as U.S. Pat. No. 4,778,758, describe and claim a device for the determination of microorganisms consisting of a sealed rectangular container containing a transparent non porous strip for the determination of the test (see claim 1 page 4, column 4, third last line).

### BRIEF SUMMARY OF THE INVENTION

The object of the present invention is to determine the Minimum Inhibitory Concentration (M.I.C.) according to the method claimed by patent No. EP 0157 071, using a paper strip printed with a known numerical sequence, corresponding to a different grading of the impregnated antibiotic, so as to simplify its extraction from the blister or the wrapping which normally protects it and avoid the creation of air bubbles between the microbial cultural medium and the paper strip itself, which could invalidate the test or render it inaccurate.

Another object of the present invention is to have a paper strip impregnated with antibiotics according to a graded scale that has the characteristic of gradually releasing the antibiotics once positioned in the microbial culture medium, so as to facilitate its repositioning on the microbial culture medium. Another object of the present invention is to reduce the cost of the method for determining the Minimum Inhibitory Concentration (M.I.C).

These and other objects are achieved by the invention that is the object of the present application which concerns the sector of chemical, clinical, bacteriological and immunological analyses and, more specifically that of the determination of the Minimum Inhibitory Concentration (M.I.C.) according to the method claimed by patent No. EP 0 157 071 using a paper strip impregnated with a predetermined concentration gradient of the antimicrobial agent according to the known technique, with a view to simplifying the execution of the relative test due to the fact that the said strip of paper is thicker than the strip of plastic and it is permeable to air and possesses the characteristic of releasing the antibiotics very slowly once positioned on the microbial culture medium, thereby facilitating its repositioning, if required, on the microbial culture medium.

### BRIEF DESCRIPTION OF THE DRAWINGS

Further features and advantages of the invention shall be better understood from the description of a preferred but not exclusive embodiment, illustrated by way of indicative but non-limiting example in the accompanying drawings in which:

FIG. 1 represents the paper strip impregnated with the predetermined concentration gradient of the antibacterial agent, graded on a colour scale of fifteen dilution intervals expressed in μg/mL according to the known technique.

FIG. 2 represents the configuration of the zone created by the placing of the aforementioned paper carrier on the microbial culture medium, the lower part of which, at the intersection of the zone with the strip, indicates the inhibiting concentration of antibiotic required to be administered to the patient.

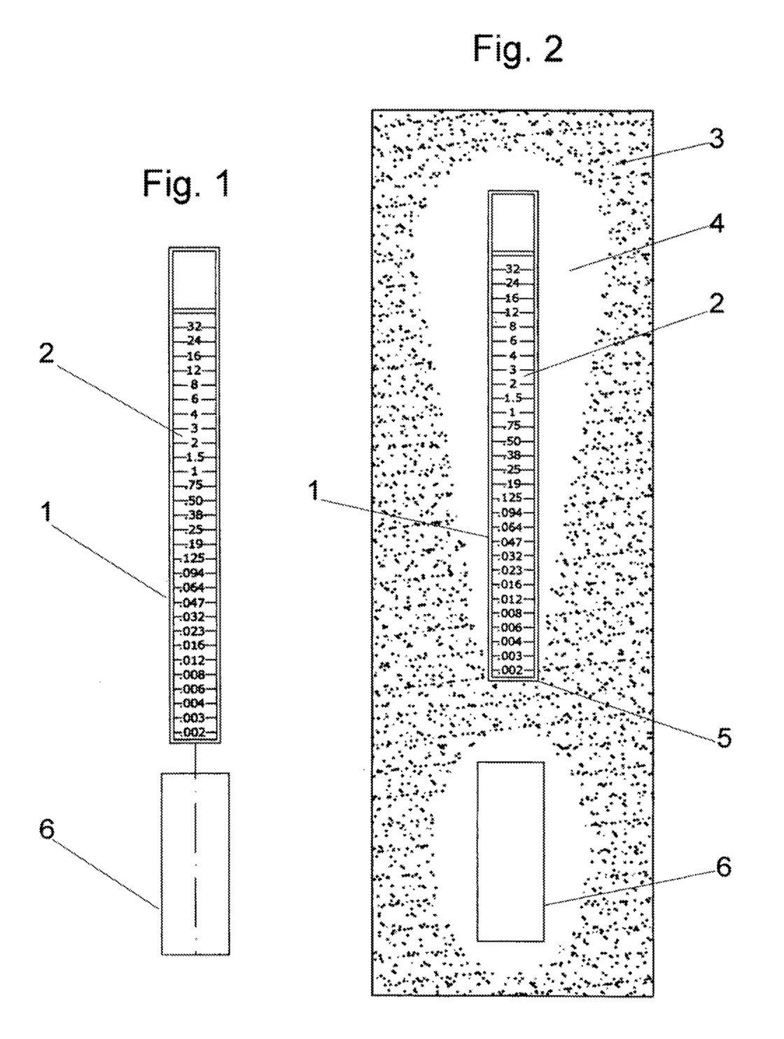

### DETAILED DESCRIPTION OF THE INVENTION

The invention that is object of the present application concerns a paper strip (1) impregnated with a predetermined concentration gradient of an antibacterial agent graded on a colour scale of fifteen dilution intervals expressed in μg/mL (2) according to the method claimed in patent EP 0 157 071 which determines the Minimum Inhibitory Concentration (M.I.C.) at a zone (4) that intersects the strip (1), positioned on a microbial culture medium of bacteria (3) taken from the patient, at point (5) at a given value reading of the antibacterial agent, and facilitates its extraction from the relative blister or wrapping thanks to its greater thickness compared to that of the strips currently in use today which, being made of thin strips of plastic, are characterized by a reciprocal force of electrostatic attraction.

Additionally, the aforementioned paper strip (1) possesses the characteristic of being permeable to air thereby preventing air bubbles from forming at the point of contact between the microbial culture medium (3) and the paper strip (1) that could invalidate the test or render it inaccurate.

A further advantage for the user is that the paper strip (1) possesses the additional characteristic that when it first comes into contact with the microbial culture medium (3) it begins to release the antibiotics with which it is impregnated very slowly, thereby allowing its user to reposition it easily on the microbial culture medium (3) for the determination of the Minimum Inhibitory Concentration (M.I.C.).

With a view to preserving from moisture the predetermined concentrations of the antibacterial agent, shown on the colour scale of fifteen dilution intervals expressed in \ig/mL (2), the paper strip (1) is equipped with a silica gel container (6).

The materials and the dimensions of the above-described invention, illustrated in the accompanying drawings and later claimed, may be varied according to requirements. Moreover, all the details may be replaced by other technically equivalent ones without for this reason straying from the protective scope of the present invention patent application.

1. Paper strip for the determination of the Minimum Inhibitory Concentration (M.I.C.) according to the method of patent No. EP 0 157 071 the carrier of which with a concentration gradient graded in fifteen dilutions according to the known technique is positioned on a culture medium of microorganisms taken from the patient and characterized by the fact that it is made of paper.

2. Paper strip for the determination of the Minimum Inhibitory Concentration (M.I.C.) according to the method of patent No. EP 0 157 071 and as claimed in claim 1 characterized by the fact of having a greater thickness compared to that of the plastic carrier used up until now so as to facilitate its extraction from the blister or wrapping that protects it.

3. Paper strip for the determination of the Minimum Inhibitory Concentration (M.I.C.) according to the method of patent No. EP 0 157 071 and as claimed in claim 1, characterized by the fact that the paper with which the said strip is made is permeable to air thereby preventing air bubbles from forming at the point of contact with the microbial culture medium that could invalidate the test or render it inaccurate.

4. Paper strip for the determination of the Minimum Inhibitory Concentration (M.I.C.) according to the method of patent No. EP 0 157 071 and as claimed in claim 1, characterized by the fact that the aforementioned paper strip on initial contact with the microbial culture medium begins to release the antibiotics with which it is impregnated very slowly and gradually facilitating the user should the strip require repositioning on the microbial culture medium.

5. Paper strip for the determination of the Minimum Inhibitory Concentration (M.I.C.) according to the method of patent No. EP 0 157 071 and as claimed in claim 1, characterized by the fact that it is equipped with a silica gel container in order to protect from moisture the predetermined concentrations of the antibacterial agent shown on the colour scale of fifteen dilution intervals expressed in μg/mL.
